(19) United States
(12) Patent Application Publication
(10) Pub. No.: US 2022/0265292 A1
(43) Pub. Date: Aug. 25, 2022

(54) METHOD AND APPARATUS FOR DELIVERY OF CELL THERAPIES

(71) Applicant: Stemplant LLC, Camden, NJ (US)

(72) Inventors: Judith T. Carpenter, Moorestown, NJ (US); Jeffrey P. Carpenter, Moorestown, NJ (US); Spencer Brown, Mt. Laurel, NJ (US); Jack B. Sattell, Ashland, MA (US); David A. Rezac, Ashland, MA (US); Devin H. Marr, Ashland, MA (US); Camron Garrett, Ashland, MA (US)

(21) Appl. No.: 17/636,752

(22) PCT Filed: Aug. 31, 2020
(86) PCT No.: PCT/US2020/048832

§ 371 Date: Feb. 18, 2022

Related U.S. Application Data
(60) Provisional application No. 62/894,862, filed on Sep. 2, 2019.

(51) Int. Cl. A61B 17/22; A61B 17/3207; A61M 25/10

(52) U.S. Cl. CPC A61B 17/22; A61B 17/320725; A61M 25/1011; A61M 25/1002; A61B 2017/22054; A61B 2017/22065; A61B 2017/00893

(57) ABSTRACT

A method and apparatus for delivery of cell therapies, introduced via percutaneous access to the circulation, and delivered to the site of vascular injury or intervention.

### CROSS-REFERENCE TO RELATED APPLICATIONS

This application claims priority under 35 U.S.C. § 119(e) to U.S. Provisional Application No. 62/894,862, filed on Sep. 2, 2019, the disclosures of which is hereby incorporated by reference herein in its entirety and made a part of the present specification. Any and all applications for which a foreign or domestic priority claim is identified in the Application Data Sheet as filed with the present application are hereby incorporated by reference under 37 CFR § 1.57.

### BACKGROUND

Cardiovascular disease is the leading cause of mortality worldwide. Atherosclerosis can lead to symptomatic blockages of major coronary arteries resulting in angina or myocardial infarction. The most common treatments include bypass surgery, atherectomy, and balloon angioplasty combined with implantation of stents. Intimal hyperplasia is the most common failure mode for atherectomy, angioplasty, and stenting. This major problem occurs after all percutaneous coronary interventions resulting in restenosis of the involved coronary arteries at rates as high as 15-30% within the first year. Intimal hyperplasia consists of an accumulation of vascular smooth muscle cells that migrate to the intimal space as well as a deposition of extracellular matrix material. The result is a decrease in the luminal diameter of the affected vessel with resultant ischemia of the end organ. The importance of treating the causes of intimal hyperplasia to prevent restenosis is evident in its prevalence and in the myriad of devices and treatments that have been proposed to address the problem.

The initial treatments involved attempts at preventing restenosis after angioplasty due to intimal hyperplasia by the development of stents. These treatments failed due to intimal hyperplasia growing through the holes in the stents. Covered stents were then developed to prevent this, but intimal hyperplasia developed at the ends of the covered stents causing restenosis. More recent treatments have attempted to address particular steps or factors in the process of intimal hyperplasia. Their lack of success can generally be attributed to the complex mechanisms involved in the development of intimal hyperplasia and to the difficulty in delivering effective treatment. The causes of intimal hyperplasia include hemodynamic factors such as shear stress and wall tensile stress, injury including endothelial denudation and medial tearing, inflammation, and genetic factors. Each of these causes involves complex pathways and a variety of cells and chemical mediators. Numerous drug therapies have been developed to attempt to decrease the development of intimal hyperplasia by targeting a specific step or cell in a particular pathway, or minimizing the initiating cause.

Current treatments to reduce intimal hyperplasia include the use of drug eluting stents and drug eluting angioplasty balloons. Some of these stents and balloons are coated with various drugs that transfer from the stent or balloon surface by direct contact with the site of the angioplasty or intervention. Others allow release of the drug near the vessel wall. The drug then contacts the vascular tissue and exerts its inhibitory effect on the hypertrophic scarring reaction (intimal hyperplasia) with the goal of decreasing the likelihood of a recurrent blockage at the treatment site.

There are several examples of devices for delivery of drugs to blood vessels. Among these are U.S. Pat. No. 5,087,244 to Wolinsky, et al.; U.S. Pat. No. 5,985,307 to Hanson, et al.; and U.S. Pat. No. 7,985,200 to Lary, et al., each of which are hereby incorporated by reference in their entireties.

Another critical vascular disease state is ruptured aneurysms, which are a leading cause of death worldwide. At present, treatment is surgical excision or ablation; there is no medical therapy for prevention or arrest of this disease. The pathophysiology of aneurysm disease has been demonstrated to be arterial degeneration, having a significant inflammatory component. The inflammatory process is located in the arterial wall and the surrounding periadventitial fat and tissues.

Some aspects of preparation and use of adipose-derived stem cells has been described in U.S. Pat. No. 8,691,216 to Fraser, et al., and U.S. Pat. No. 9,198,937 to Fraser, et al. in which such stem cells are used to promote wound healing and liver injury by delivery via a catheter equipped with a balloon.

Recently it has been shown that intimal hyperplasia can be reduced by the introduction of stem cells to the site of angioplasty-induced arterial injury from outside the vessel. It is also believed that stem cell therapies may be a candidate medical therapy for aneurysms. No delivery systems for cellular therapies are currently available or appropriate for treatment of aneurysm disease. The fragility of the affected arterial wall presents special challenges for delivery of therapies with the risk of manipulation of the aneurysmal tissue at the time of treatment.

Finally, it is believed that stem cell therapies may be used as medical treatments for other disease states, provided that cellular suspensions may be delivered directly in close proximity to the tissue needing treatment, and delivery via the circulatory system using percutaneous access is a desired method.

Devices for delivery of cell therapies to the lumen of the vessel for this type of application have not been developed or commercialized. A difference between current drug-coated interventional devices and cell therapies is that to be viable, stem cells must be freshly prepared and maintained in suspension (as opposed to coated on the interventional apparatus and maintained in a dry state for storage). A further difference is that stem cells (and other cellular suspensions) are larger in size compared to drug molecules, and would be swept away from the interventional site by blood flowing though the vessel after the procedure is completed. Thus, a new method of delivering a liquid suspension of properly prepared fresh stem cells or non-stem cells to the site of the intervention, and retaining them there is required.

### SUMMARY

In some embodiments, disclosed herein is a method and apparatus for delivery of therapeutic agents, including but not limited to cell and non-cell based therapies, drugs, growth factors, and the like, introduced via percutaneous access to the circulation, and delivered to the site of vascular injury or intervention or to the surrounding tissue. The cellular suspensions are delivered to the intima, subintimal space, media, adventitia, or periadventitial space from within the lumen of the vessel or to periarterial tissues and fat from within the lumen of an adjacent vessel.

In some embodiments, systems and methods can be utilized to deliver stem cell therapy, including but not limited to mesenchymal stem cell (MSC) therapy beneath the endothelium of a vessel to reduce intimal hyperplasia after angioplasty. Mesenchymal stem cells (MSCs) and/or progenitor or precursor cells can be isolated from a variety of tissues, such as bone marrow, skeletal muscle, dental pulp, bone, umbilical cord, amniotic fluid, and adipose tissue. The MSC therapy could include a stromal vascular fraction. In some embodiments, MSCs can include any number of stem cells, mesenchymal stem cells, marrow stromal cells, multipotent stromal cells, and/or multipotent stem cells and be derived from various adult tissues. In some embodiments, MSCs are isolated from Wharton's jelly. In another embodiment, MSCs are derived from iPS cells. In some embodiments, the MSCs include autologous, homogenous, non-manipulated adult MSCs. In some embodiments, systems and methods can include bone marrow mesenchymal stem cells (BM-MSC), including but not limited to allogenic passage 2 BM-MSCs which can be cryopreserved and thawed the day of the protocol. In some embodiments, a therapy can include any number of autologous bone marrow aspirates; bone marrow derived aldehyde dehydrogenase bright cells (ALDHbr); combination bone marrow MSC CD34+ plus bone marrow derived endothelial precursor cells; human placental cells (e.g., PLX-PAD from Pluristem and PDA-002 from Celularity Inc.) and/or endometrial regenerative cells (ERC). In some embodiments, one advantage of allogenic bone marrow MSCs are that cryo-preserved cells can be prepared prior to any protocol and that a second surgical procedure would not have to be performed to obtain and prepare fresh autologous adipose stromal vascular fractions.

In some embodiments, a gel foam can be utilized to deliver the therapy, with or without placement on a catheter including an expandable member, such as a balloon.

In some embodiments, systems and methods include a cell-based therapy, but does not include a drug. In some embodiments, a system or method does not include any drugs, such as anti-neoplastic agents, e.g., paclitaxel. In some embodiments, an expandable member, such as a balloon, is not coated by, or does not otherwise include any drugs, such as anti-neoplastic agents, e.g., paclitaxel.

In some embodiments, a method can include accessing a target vessel; performing an angioplasty procedure; and delivering the therapeutic agent to the site of vascular injury. Not to be limited by theory, but if cell delivery conduits are co-located with stress concentrating elements, the therapy can be delivered preferentially to sites of greater need. In some embodiments, systems and methods can includes structural features and/or functions of conventional scoring balloons or other balloons. In some embodiments, a balloon catheter can be utilized for use in a body lumen, e.g., the peripheral artery vasculature, to deliver cell-based therapies (including but not limited to, e.g., autologous adipose stromal vascular fraction or autologous/passage 2 cultured allogenic bone marrow mesenchymal stem cells) directly to a target lesion in order to promote positive remodeling in sub-intimal tissue.

Intimal hyperplasia is a response to injury inside arterial walls. Use of various drugs (paclitaxel) have been shown to decrease the inflammatory response and proliferation of the smooth muscle cells. The released drugs decrease over time and may only be effective in a short time in the wound healing scenario. Not to be limited by theory, in some embodiments, an adult mesenchymal cell-based approach can be superior to a pharmaceutical approach for modulating the intimal hyperplasia response. The direct delivery of viable immunomodulatory stem cells into the arterial wall can in some cases provide a much longer sustained anti-inflammatory and regenerative effect than a drug. Mesenchymal stem cells secrete anti-inflammatory factors, growth factors and cell signaling factors. As the injured arterial wall heals, the demands for immunomodulation and regeneration change over time and the retained stem cells can be advantageously reactive.

### BRIEF DESCRIPTION OF THE DRAWINGS

FIG. 1 depicts a cross-section view of an embodiment of the delivery device of the present invention.

FIG. 2A schematically illustrates an expandable member, such as an inflatable medical balloon that can be wrapped with one or more structures.

As illustrated in FIG. 2B, if wider tubes can be used, the apertures can also be wider to allow for improved flow.

As shown in FIG. 2C the tubes can include elevated rims around the apertures, and/or small needles extending radially outward as illustrated.

FIG. 3A illustrates a flow-through non-occlusive balloon embodiment that can advantageously allow continuous blood flow through the vessel to be treated, in order to allow more time for therapeutic injection.

FIG. 3B illustrates an embodiment of a multi-lobed balloon with holes or penetrating members (e.g., spikes or needles) where the expanded balloon lobes contact the luminal wall.

FIG. 4A schematically illustrates a method of delivering a therapeutic agent, such as a stem cell therapy for example, to a target luminal site.

As illustrated schematically in FIG. 4B, delivery devices can include barriers, for example, at the proximal and/or distal ends of the device to prevent the therapeutic agent (e.g., stem cell mixture) from migrating out of the target therapeutic area, and allowing for the stem cells to move into the vessel wall.

FIG. 5A schematically illustrates an embodiment of a delivery device including an outer balloon and an inner balloon.

FIG. 5B schematically illustrates various embodiments of delivery features of the outer balloon, including holes, sphinctered holes (that can transform from an empty, closed relaxed state to an open, full state under pressure), and/or spiked injection needles that can have either straight or curved (including helical) geometries.

FIG. 6 schematically illustrates a delivery device including an expandable member and a pad adhered to the expandable member and configured to secure an infusion tube.

FIGS. 7A-7B schematically illustrate views of a balloon-in-balloon device including an outer balloon and an inner balloon.

FIGS. 8A-8C schematically illustrate views of a delivery tube that can wrap around the expandable member in a helical configuration.

FIG. 9 schematically illustrates a delivery system that can be utilized, for example, in any configuration wherein the cell infusion tubes are stationary during the injection process.

FIGS. 10A-10C schematically illustrate delivery device embodiments where the infusion tubes are not fixed with respect to the balloon, but can be pulled in a desired direction, e.g., proximally or distally, by the user.

FIG. 11 schematically illustrates delivery device embodiments where the infusion tubes are held and organized by a unifying structure, such as, for example, a stent-like self-expanding cage.

FIGS. 12A-12C schematically illustrates a delivery device embodiment where an array of ported infusion tubes would be delivered over a guidewire first.

FIGS. 13A-13B schematically illustrates a delivery device embodiment where the infusion lumens are created by splitting a hypotube into a number of segments that join into full tubes at both ends.

FIGS. 14A-14B schematically illustrates a delivery device embodiment where the cell infusion tubes could be made of a compliant material.

FIG. 15 schematically illustrates a delivery device embodiment where the balloon could have cutting or scoring elements, such as atherotomes, each sheathed in their own compliant tube.

FIG. 16 schematically illustrates a delivery device embodiment including a balloon-in-balloon where the inner balloon is configured to apply the pressure for angioplasty, and the outer balloon is configured to provide a lumen for MSC infusion.

FIGS. 17A-17B schematically illustrates a delivery device embodiment including a balloon including scoring elements configured to have a curved cross section that forms an open channel when opposed to the vessel wall.

FIGS. 18A-18B schematically illustrates a delivery device embodiment that can be similar to that of FIGS. 17A-17B described above, where the therapeutic agent (e.g., MSC suspension) can be delivered at or proximate to the proximal end of the full diameter inflated balloon (e.g., via inflation ports) and can be distributed along the full lesion length by the channel created by the scoring elements.

FIGS. 19A-19B schematically illustrates a delivery device embodiment where the infusion tubes are not bonded to the balloon, but are oriented and constrained radially by elongate structures, such as, for example, wires running through their centers.

FIG. 20 schematically illustrates a delivery device embodiment where the therapeutic agents, e.g., the MSCs, can be preloaded into the spaces underneath the balloon pleats.

FIG. 21 schematically illustrates a delivery device embodiment including a single inflation lumen made from a laser cut hypotube.

FIG. 22 schematically illustrates a delivery device embodiment including dual expandable members.

FIGS. 23A-23B schematically illustrates a delivery device embodiment and injection method.

### DETAILED DESCRIPTION

In some embodiments, disclosed herein is an expandable member, including but not limited to a balloon, that carries on its surface one or more tubes having apertures or a continuous groove. Once inflated to fracture the plaque layer, the cellular suspension is introduced through the tube where it flows from the apertures (or groove) into the vessel wall. Some embodiments may be equipped with a central passage to permit blood flow during inflation. The medium in which the cells are suspended may be a biologically neutral or active solution, and may optionally comprise drugs, biological agents, and other additives.

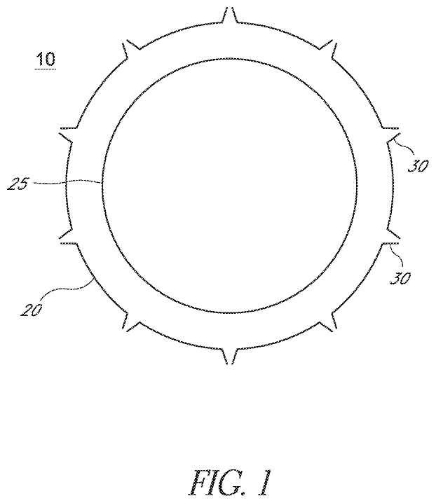

One non-limiting embodiment as disclosed in FIG. 1 includes a toroidal balloon 10, having hollow rigid spikes 30 on its outer surface 20. This balloon is attached to a conventional catheter (not shown) for percutaneous access to a vessel under treatment and is positioned in the vessel in an uninflated or minimally inflated state. The inner wall 25 of the toroidal balloon may be made of relatively stiff material to facilitate insertion and placement of the balloon. Once in place, the balloon may be inflated by the introduction of a cellular suspension under pressure, which both inflates the balloon 10, drives the hollow rigid spikes 30 into and through the plaque layer, and delivers the suspension through the spikes 30 into the plaque and/or the desired structure of the vessel wall. The balloon 10 is then deflated and withdrawn, leaving the stem cells behind, but protected from being swept away in the blood flow. Because the balloon 10 is toroidal, blood flow through the vessel under treatment is never interrupted.

In an alternative embodiment (not shown), balloon 10 may be spherical or elongated, but not toroidal, and during inflation, will interrupt blood flow through the vessel under treatment. Balloon 10 may optionally be provided with annular flanges (not shown) at the proximal and distal ends. These flanges engage the vessel wall, and serve to prevent blood flow between the vessel wall and the outer surface 20, instead directing blood flow through the torus.

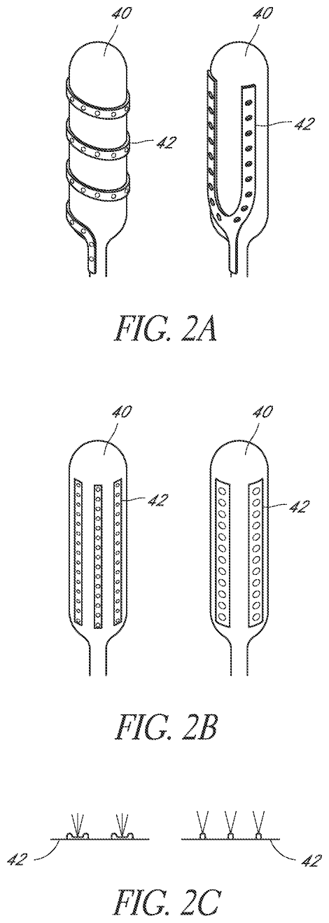

FIG. 2A schematically illustrates an expandable member 40, such as an inflatable medical balloon that can be wrapped with one or more structures 42. The wrapped balloon can differ from scoring balloons in that instead of wires, there can be hollow tubes with apertures 42. The tubes 42 can be made of metal or another desired material. The tubes 42 can include lumens of various cross-sections, including round, semi-circular (e.g., half moon), or flattened geometries. The hollow tubes can be oriented in various directions with respect to a longitudinal axis of the balloon, such as oblique/helically wrapped, or aligned with the longitudinal axis of the balloon as shown. In some embodiments, the hollow tubes can be placed transverse to the longitudinal axis of the balloon. The expandable member 40 can be a balloon with a balloon lumen. There can be a separate injection port for the tubes 42. The proportions between the expandable member 40 and the one or more structures 42 can vary.

As illustrated in FIG. 2B, if wider tubes 42 can be used, the apertures can also be wider to allow for improved flow. The comparison of FIG. 2 shows wider tubes 42 on the right with wider apertures for improved flow. For example, the apertures can be elongate and include a dimension (e.g., width) greater than that of another dimension (e.g., length), such as about or at least about 10%, 25%, 50%, 75%, 100%, or more greater in a second dimension relative to a first dimension. As shown in FIG. 2C the tubes 42 can include elevated rims around the apertures, and/or small needles extending radially outward as illustrated.

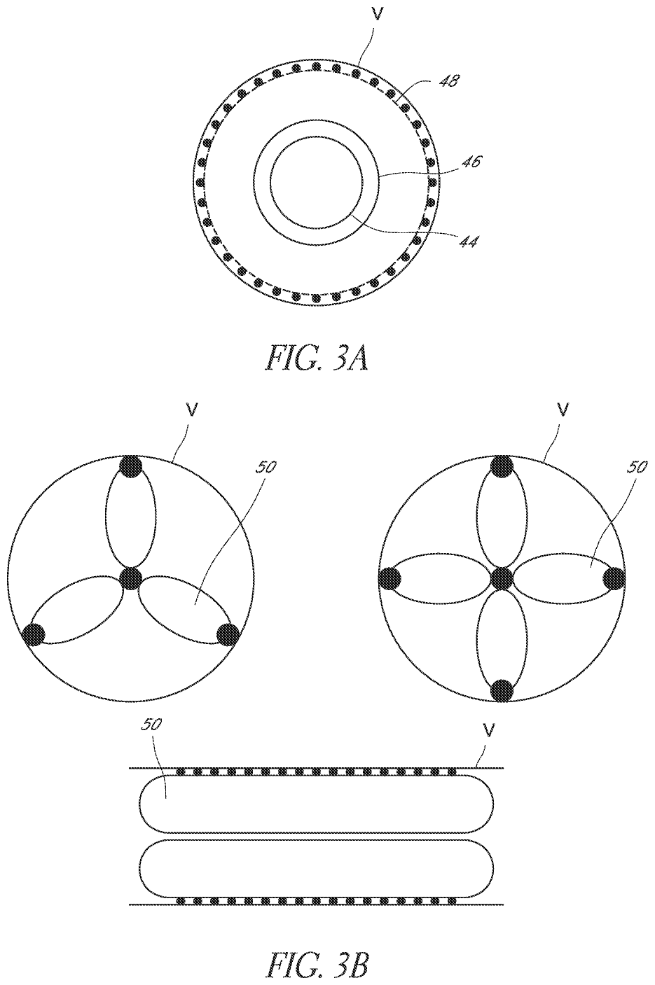

FIG. 3A illustrates a flow-through non-occlusive balloon embodiment that can advantageously allow continuous blood flow through the vessel to be treated, in order to allow more time for therapeutic injection. FIG. 3A illustrates the vessel wall V. Positioned within the vessel wall is an open central tube 44, an inner balloon 46, and an spiked or porous outer balloon 48. A delivery device can include an inner catheter/tube 44 and an outer expandable member, such as a multi-layer balloon (e.g., dual lumen balloon 46, 48 as shown).

The balloons 46, 48 can be mounted on the inner tube 44 which remains open and in fluid communication with the body lumen to allow flow (e.g., blood flow) therethrough. The outer balloon 48 can include conduits, such as holes (e.g., sphinctered holes) and/or penetrating members (e.g., spikes or needles). The penetrating members can include conduits therethrough. The delivery devices can accomplish delivery of the therapeutic agent (e.g., stem cell mixtures and/or drugs alone), or be used to accomplish both an angioplasty procedure and delivery of the therapeutic agent, such that a separate angioplasty balloon is not required.

FIG. 3B illustrates an embodiment of a multi-lobed balloon 50 with holes or penetrating members (e.g., spikes or needles) where the expanded balloon lobes contact the luminal wall V. The multi-lobed balloon 50 can include any number of lobes (e.g., two, three, four, five, six, or any range of the foregoing values). Each lobe can include any number of holes or penetrating members (e.g., zero holes, one hole, two holes, three holes, zero penetrating members, one penetrating member, two penetrating members, three penetrating members, or any range of the foregoing values).

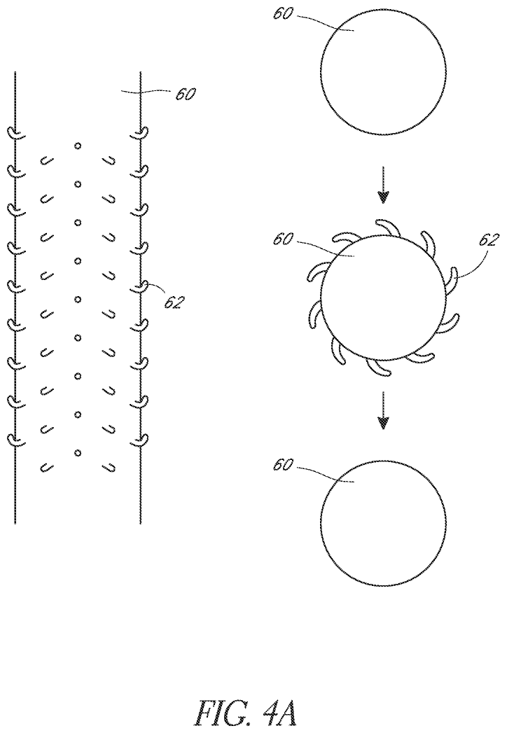

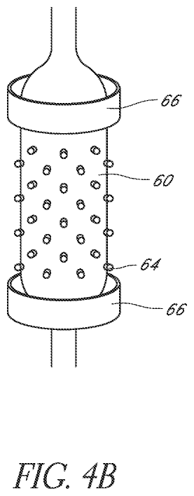

FIG. 4A schematically illustrates a method of delivering a therapeutic agent, such as a stem cell therapy for example, to a target luminal site. A delivery device 60 can be placed at an angioplasty site following an angioplasty (or alternatively a delivery device 60 can perform both the angioplasty, and the therapeutic agent delivery as discussed elsewhere herein). The therapeutic agent 62 can then be injected into the lumen, and the delivery device can then be removed. In some embodiments, the therapeutic agent 62 can include a stem cell mixture or drug.

In some embodiments, as illustrated schematically in FIG. 4B, catheters 60 can include needles 64 that move outward, e.g., radially outward, and into the vessel wall for delivery of the therapeutic agent, and move inward, e.g., radially inward following delivery of the therapeutic agent for removal. The needles 64 can moved outward and inward via a screw mechanism, e.g., rotating along threads for example.

In some embodiments, a delivery device includes a catheter with apertures with or without raised edges or flanges that otherwise crank or actuate open to contact the luminal wall and deliver a therapeutic agent (e.g., stem cell mixture).

As illustrated schematically in FIG. 4B, delivery devices 60 can include barriers 66, for example, at the proximal and/or distal ends of the device to prevent the therapeutic agent 62 (e.g., stem cell mixture) from migrating out of the target therapeutic area, and allowing for the stem cells to move into the vessel wall. The barriers 64 can include expandable members such as inflatable rings, for example.

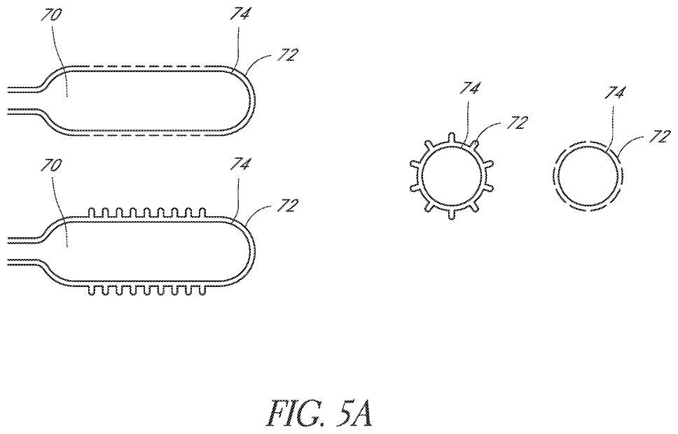

FIG. 5A schematically illustrates an embodiment of a delivery device 70 including an outer balloon 72 and an inner balloon 74. The outer balloon 72 can include small holes, sphinctered holes, and/or spiked needles concentrated in the side areas that contact the luminal wall. The inner balloon 74 can be filled with media, such as normal saline for example, to provide sufficient pressure to complete the angioplasty, then partially relaxed. A stem cell mixture (e.g., stromal vascular fraction) can be injected into the outer balloon, and then the inner balloon is repressurized to eject the stem cell mixture. Other therapeutic agents, such as drugs, can also be delivered in the same or a similar fashion. FIG. 5A illustrates two embodiments of the delivery device 70 having different outer balloons 72.

FIG. 5B schematically illustrates various embodiments of delivery features of the outer balloon 72, including holes, sphinctered holes (that can transform from an empty, closed relaxed state to an open, full state under pressure), and/or spiked injection needles that can have either straight or curved (including helical) geometries. The sphinctered holes are shown in greater detail in an empty state on the left and in a full, under pressure state on the right.

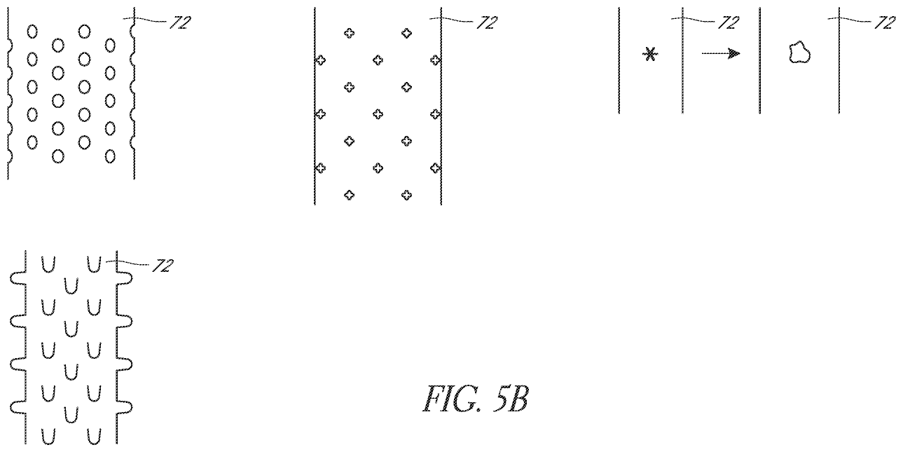

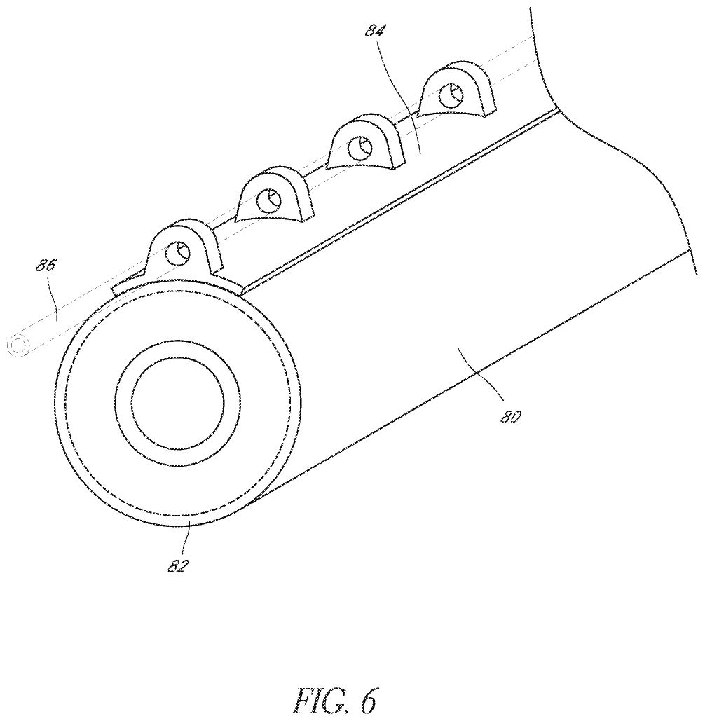

FIG. 6 schematically illustrates a delivery device 80 including an expandable member 82 and a pad 84 adhered to the expandable member 82 and configured to secure an infusion tube 86. The infusion tube can be a hypotube. The pad 84 can be flexible and conform to the outer surface of the expandable member 82 as shown, and be attached to the balloon via adhesive, for example. The pad 84 can include a series of eyelets configured to receive the infusion tube 86 therethrough. This can advantageously allow the infusion tube to be constrained and oriented on the balloon 82 without being directly attached to the balloon 82. The pad 84 can have a relatively large surface area for robust attachment to the balloon 82. The sprinkler or infusion tube 86 is not bonded

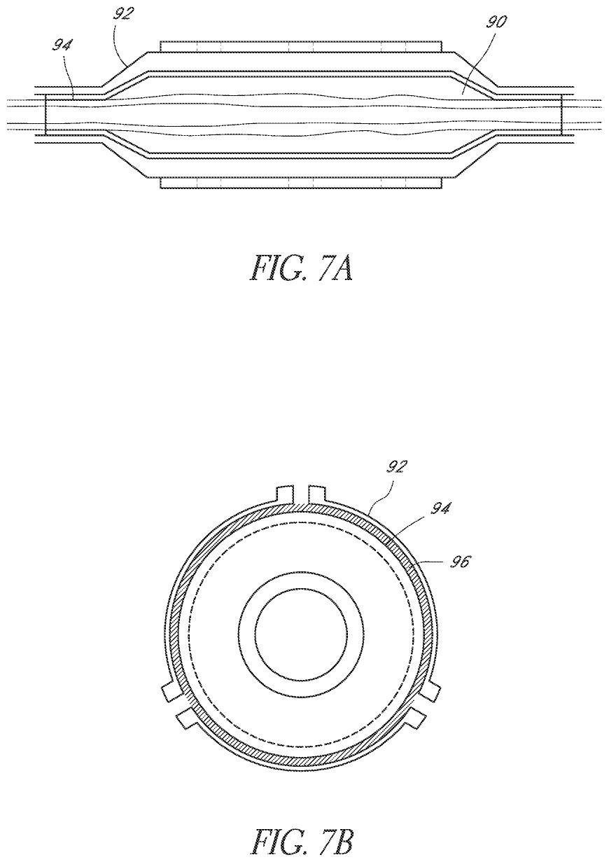

FIGS. 7A-7B schematically illustrate views of a balloon-in-balloon device 90 including an outer balloon 92 and an inner balloon 94. The inner balloon 94 can provide sufficient pressure for angioplasty, and the space 96 between the inner and outer balloons 92, 94 is used as an annular lumen to deliver cells. Perforated stress concentrators (e.g., holes or needles) attached to the outer balloon 92 can provide a path for cells to travel from the inter-balloon space to the vessel wall. The balloon-in-balloon device 90 can include a guidewire lumen.

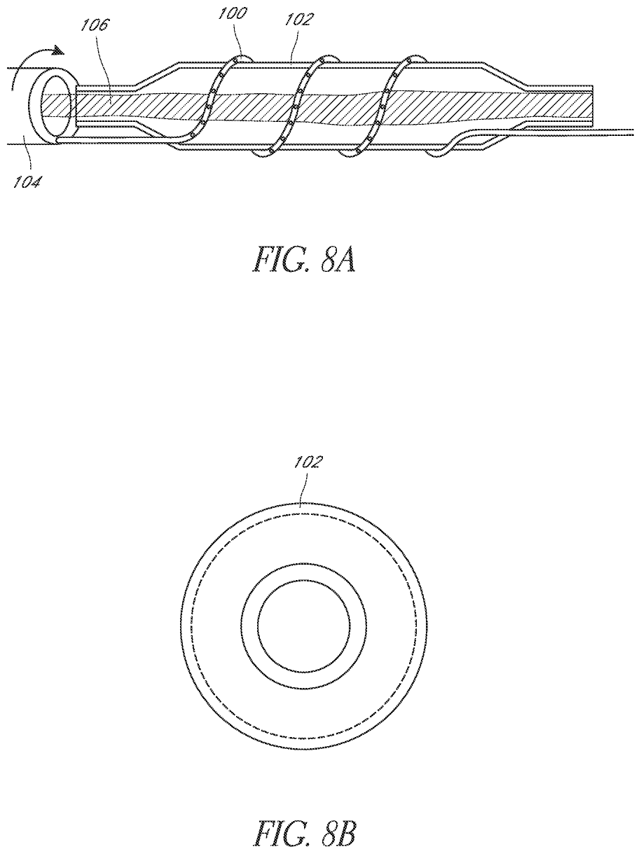

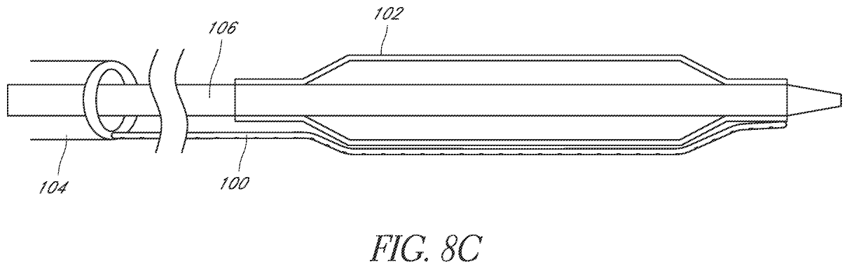

FIGS. 8A-8C schematically illustrate views of a delivery tube 100 that can wrap around the expandable member 102 in a helical configuration. The tube 100 could be in a substantially straight configuration, or be wound into a tighter helical form during delivery to decrease the overall diameter of the system and can assume an expanded coil shape after reaching the target lesion. This can be achieved, for example, either with a highly elastic pre-set material such as a shape memory material, e.g., nitinol, or by rotating one end of the delivery tube relative to a central torque-resistant shaft. There can be rotation of handle 104 attached to the delivery tube 100 to open the coil of the delivery tube 100. There can be a torque resistant shaft 106. In some embodiments, the torque resistant shaft 106 prevents rotation of the expandable member 102. The delivery tube 100 can be a lined, lasercut nitinol shapeset. The delivery tube 100 can be delivered straight as shown in FIG. 8C.

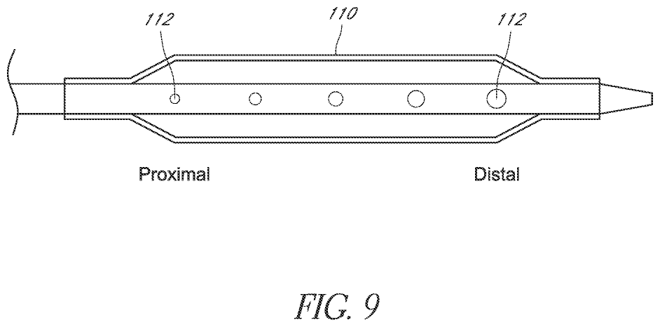

FIG. 9 schematically illustrates a delivery system 110 that can be utilized, for example, in any configuration wherein the cell infusion tubes are stationary during the injection process. To deliver a consistent volume of cells along the length of the treatment zone, the cell infusion tube can include progressive hole sizing of holes 112 (e.g., each successive hole increasing in size/diameter, for example). As the pressure in the tube can drop after each hole, having smaller holes proximally where the pressure is higher and larger holes distally where the pressure is lower could result in a more even distribution of cells.

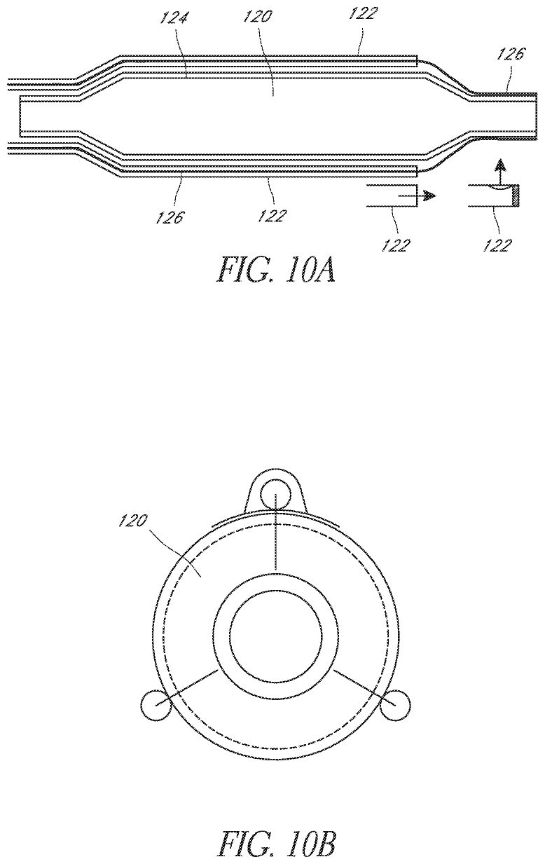

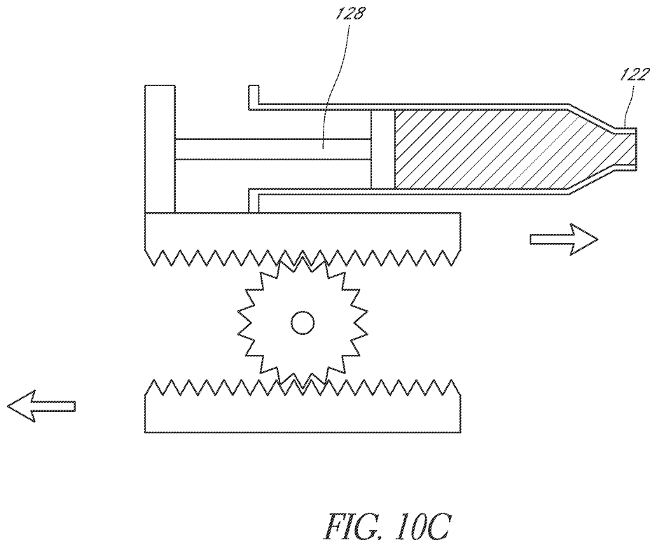

FIGS. 10A-10C schematically illustrate delivery device 120 embodiments where the infusion tubes 122 are not fixed with respect to the balloon 124, but can be pulled in a desired direction, e.g., proximally or distally, by the user. The treatment sequence can begin with the distal end of the infusion tubes lined up with the distal end of the target therapeutic zone (e.g., full diameter section of the balloon). After the balloon is inflated, the infusion tubes 122 can be pulled proximally while ejecting cells out of the distal end. The orientation of the infusion tubes 122 could be maintained either with eyelets attached to the balloon 124 or with wires 126 running through the infusion tubes 122 that are attached at the distal end of the balloon 124. The tubes 122 could have open distal ends or plugged distal ends with side ports to direct the flow of cells directly into the vessel wall. The motion to advance the plunger 128 could also be directly tied to the motion to pull the infusion tubes 122 proximally through a mechanical means such as, for example, a rack and pinion. In some embodiments, the infusion tubes 122 can be pulled back to spread cells. The enlarged view of the infusion tubes 122 shows that the flow direction can include flow out of the end or flow out the side. The wire 126 can help hold the orientation. In some embodiments, eyelets are utilized to guide the infusion tubes. The plunger 128 advancement can drive infusion tube pullback.

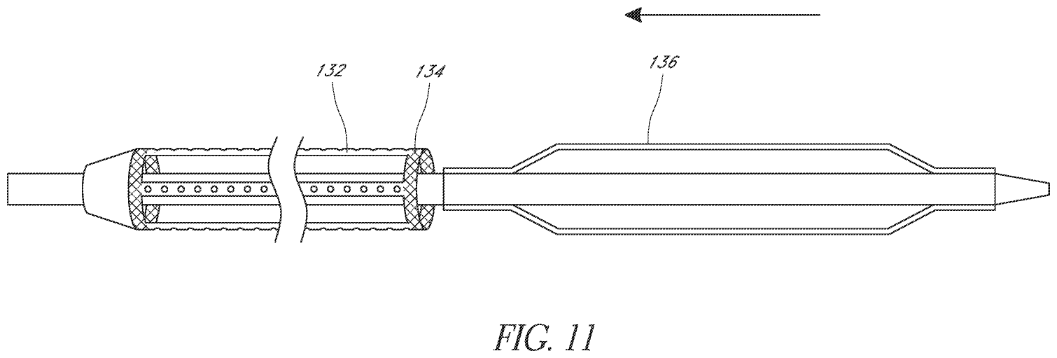

FIG. 11 schematically illustrates delivery device 130 embodiments where the infusion tubes 132 are held and organized by a unifying structure, such as, for example, a stent-like self-expanding cage 134. To reduce the profile during delivery, the balloon 136 could be coaxial but distal to the infusion cage 134. After reaching the target lesion, the balloon 136 could then be retracted into the infusion cage 134 and expanded within it. The cage 134 can include stent-like expansion struts. The cage 134 tracks behind balloon 136 to minimize bulk on outer diameter. The balloon 136 retracts into the stent link sprinkler cage 134 in the direction of the arrow. The cage 134 can spread infusion material through one or more holes or penetrators.

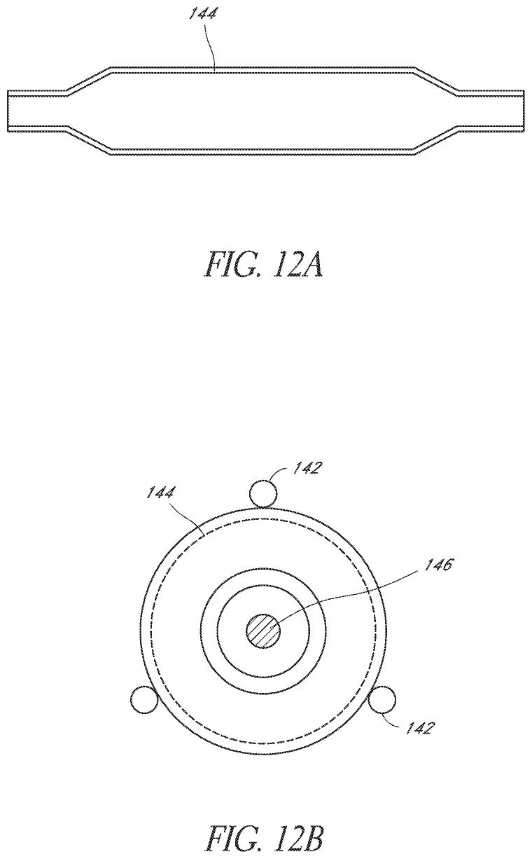

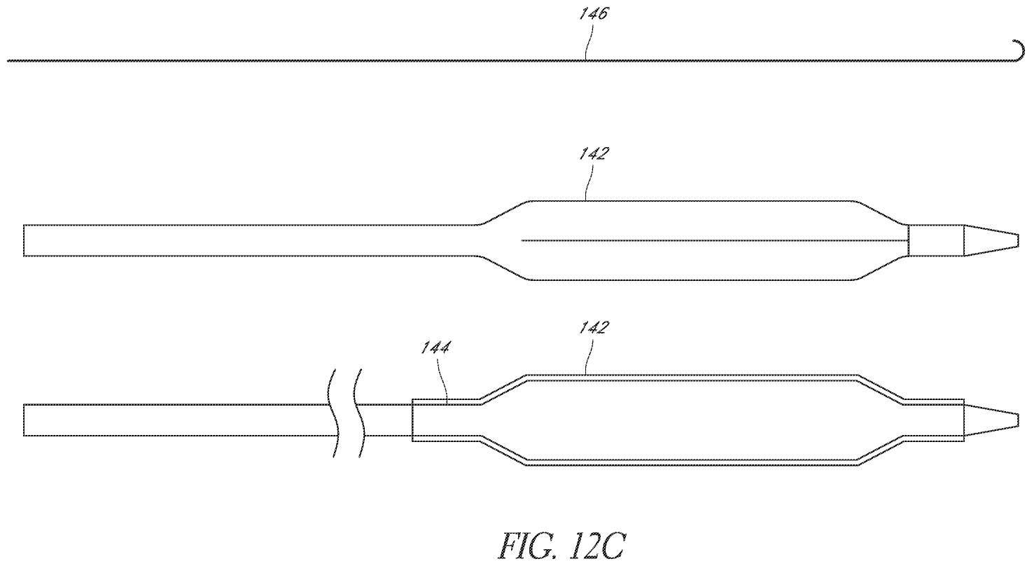

FIGS. 12A-12C schematically illustrates a delivery device 140 embodiment where an array of ported infusion tubes 142 would be delivered over a guidewire first. The array can include, for example, two, three, four, five, or more infusion tubes 142. The balloon 144 would subsequently be delivered inside (e.g., radially inward) of the array with potential for a docking mechanism to register the array with the balloon during treatment. The concept of a multi-stage deployment may in some embodiments improve overall trackability and device profile by reducing the amount of material that has to be delivered through a given sheath or lesion segment cross section during any particular procedural step. FIG. 12C shows the separate deployment stages so that the ported array 142 can be pre-deployed over a guidewire 146 with subsequent delivery of the balloon catheter 144 along the guide wire 146 inside the ported array 142. The ported array 142 can include ports that infusion tubes 142 extend through. The ported array can be any structure that allows the connection of infusion tubes 142.

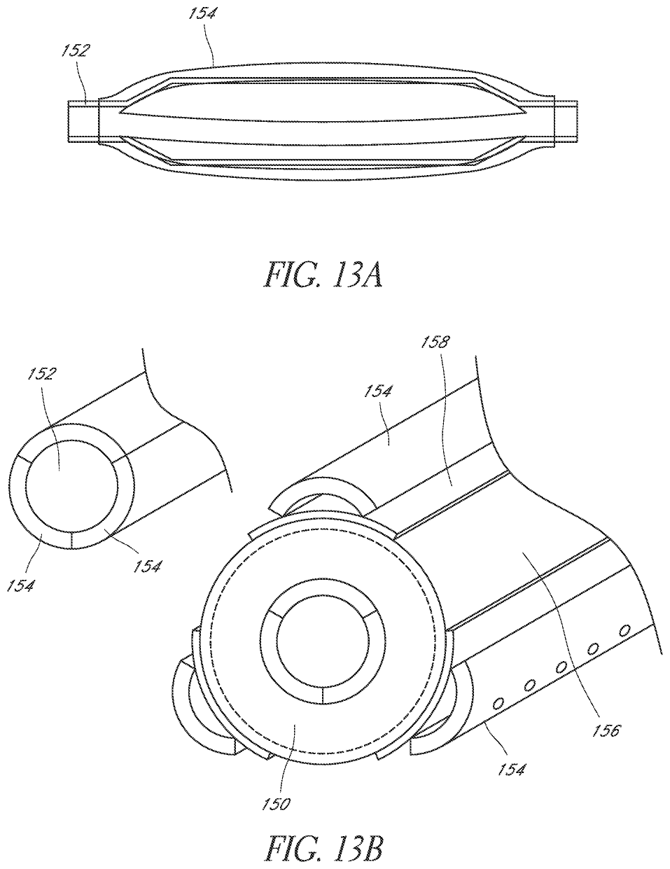

FIGS. 13A-13B schematically illustrates a delivery device 150 embodiment where the infusion lumens are created by splitting a hypotube 152 into a number of segments 154 that join into full tubes at both ends. For example, the tube 152 can include 3 semilunar segments arranged 120 degrees apart, or other numbers of segments, such as 2, 4, 5, 6, 7, 8, 9, 10, or more. The balloon 156 can be positioned and sit inside of the split segments 154 and could have compliant pads 158 to seal against the edges of the hypotube segments 154. The compliant pads 158 could have cutouts to allow for a therapeutic agent, e.g., MSCs to seep out in specific locations. The hypotube segments 154 could have holes for additional MSC delivery. The pads 158 can have a compliant sealing layer. The device can include grooves to allow targeted seepage. The segments 154 can extend the length of the balloon 156. The segments 154 can rejoin to be the full circle hypotube 152 at both ends. The full tube 152 travels to the proximal end. The full tube 152 travels to the distal end.

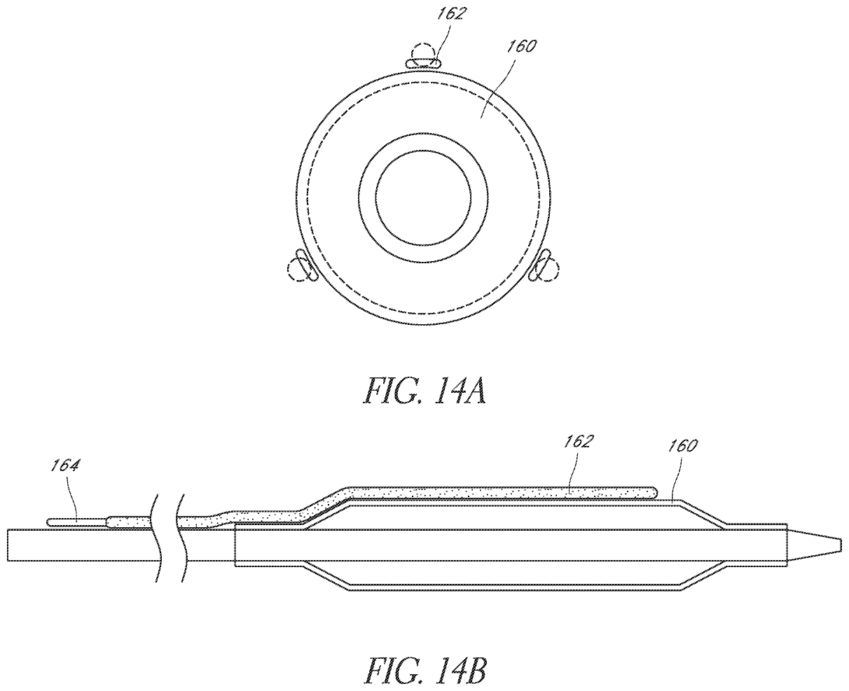

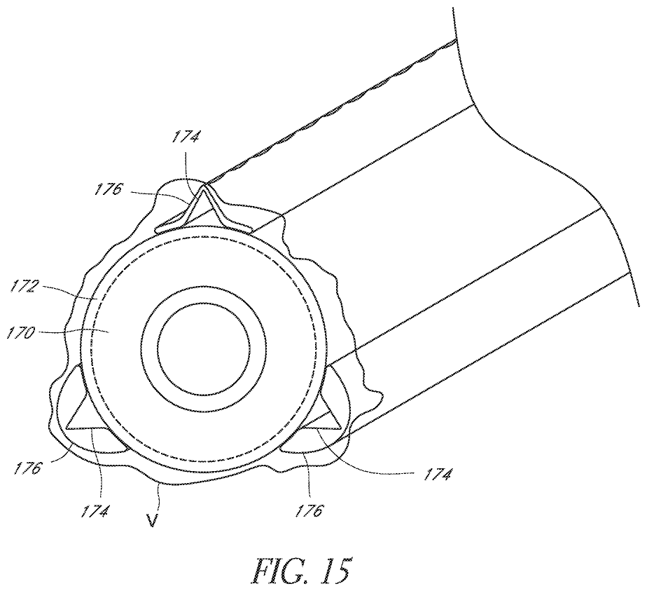

FIGS. 14A-14B schematically illustrates a delivery device 160 embodiment where the cell infusion tubes 162 could be made of a compliant material (e.g., thin-walled PET, heat shrink, ePTFE, and the like) so that they would lay flat for delivery but could then be inflated with a therapeutic agent, e.g., MSCs prior to balloon inflation. The infusion tubes could be designed with features (e.g., ported or scored features) that would allow for delivery of the therapeutic agent, (e.g., rupture at a specific pressure) to allow the release of MSCs. The infusion tubes 162 could also be filled proximally with a plunger, valve, or other element that can prevent the MSC suspension from being forced proximally by the back pressure from balloon inflation. The infusion tubes could conform to balloon surface during delivery, then inflate to dashed profile with MSC prior to balloon inflation. The lumens could be ported or scored to rupture at target pressure. The plunger 164 can be actuated to fill and support MSC suspension against back pressure. The suspension of MSC is inside delivery lumen.

FIG. 15 schematically illustrates a delivery device 170 embodiment where the balloon 172 could have cutting or scoring elements 174, such as atherotomes, each sheathed in their own compliant tube 176. That tube 176 could lay flat for delivery, but after balloon inflation could be infused with the therapeutic agent, e.g., MSCs. The tube 176 could either have perforations built in, or the force of balloon inflation could cause the scoring element 174 to pierce the tube creating infusion ports. The scoring elements 174 could either have a single straight edge or could have a series of points or high spots which could cause predictable perforation of the inflation tube. FIG. 15 shows a multi-edge version of the scoring elements 174 (near top). The arthrotomes pierce the sock or compliant tube 176 to create area of delivery. The sock or compliant tube 176 can be unpressurized, as shown near the multi-edge version. FIG. 15 shows a single edge version of the scoring elements 174 (near bottom right). The sock or compliant tube 176 can be pressurized, as shown near the bottom left scoring element 174. The vessel wall V is shown. The flow of agents such as stem cells can flow form the sock or compliant tube 176.

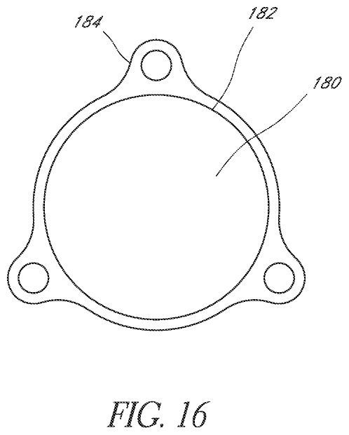

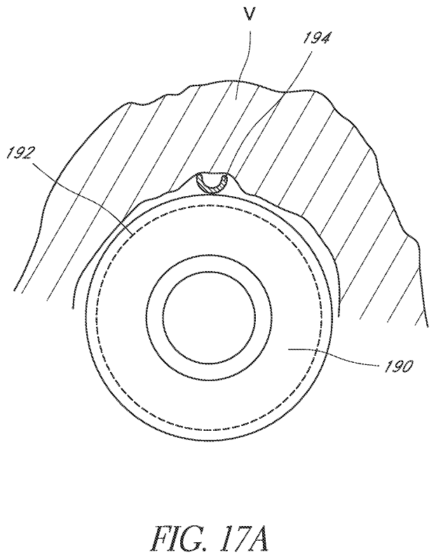

FIG. 16 schematically illustrates a delivery device 180 embodiment including a balloon-in-balloon where the inner balloon 182 is configured to apply the pressure for angioplasty, and the outer balloon 184 is configured to provide a lumen for MSC infusion. The outer balloon 184 could be an elastic sleeve or a semicompliant balloon that fits tight to the inner balloon. Additional elements in the annulus between the two balloons, such as a hypotube that has been laser cut (such as spiral cut) for flexibility, can provide stress concentrators to break plaques as well as create gaps for therapeutic agents, including MSCs, to flow down the length of the balloon. The outer balloon 184 can have a hypotube support and can be spiral cut.

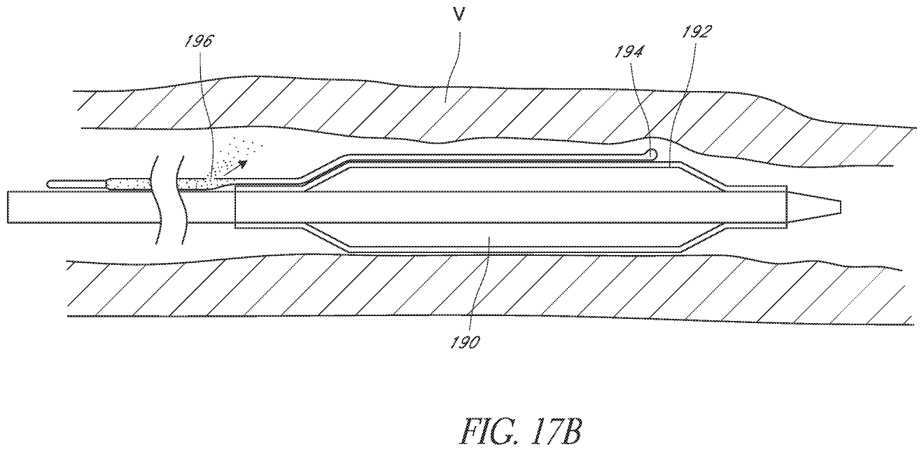

FIGS. 17A-17B schematically illustrates a delivery device 190 embodiment including a balloon 192 including scoring elements 194 configured to have a curved cross section that forms an open channel when opposed to the vessel wall. The curved cross section can be generally opposite of the general curve of the balloon in some cases. The therapeutic agent, e.g., MSC suspension, could then be delivered proximal to the inflated balloon. The delivery device 190 can include a MSC delivery lumen exit portion 196. The vessel wall V is shown. The MSC suspension can be delivered proximal to the inflated balloon 192, then carried by the bloodstream into tissue via channels of the scoring element 194.

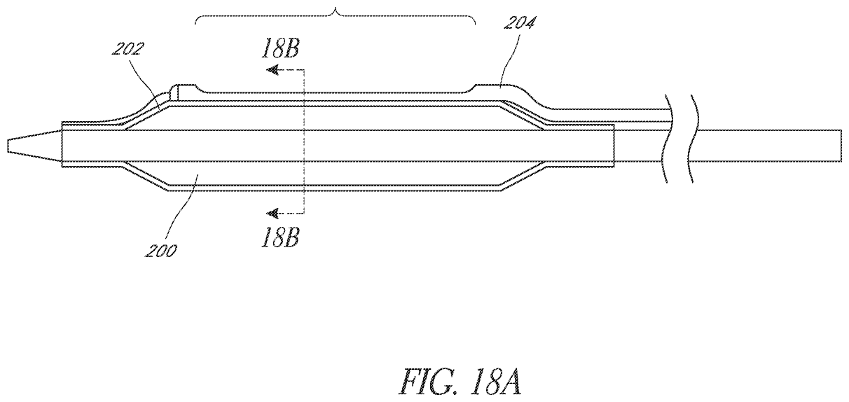

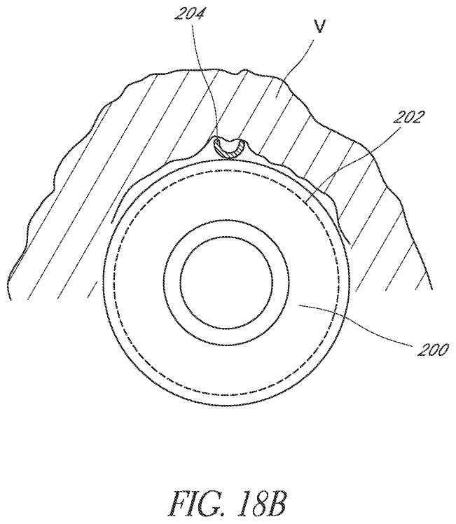

FIGS. 18A-18B schematically illustrates a delivery device 200 embodiment that can be similar to that of FIGS. 17A-17B described above, where the therapeutic agent (e.g., MSC suspension) can be delivered at or proximate to the proximal end of the full diameter inflated balloon 202 (e.g., via inflation ports) and can be distributed along the full lesion length by the channel created by the scoring elements 204. The scoring element 204 can be laser welded. The scoring element 304 can be scalloped along length of the delivery port. The vessel wall V is shown.

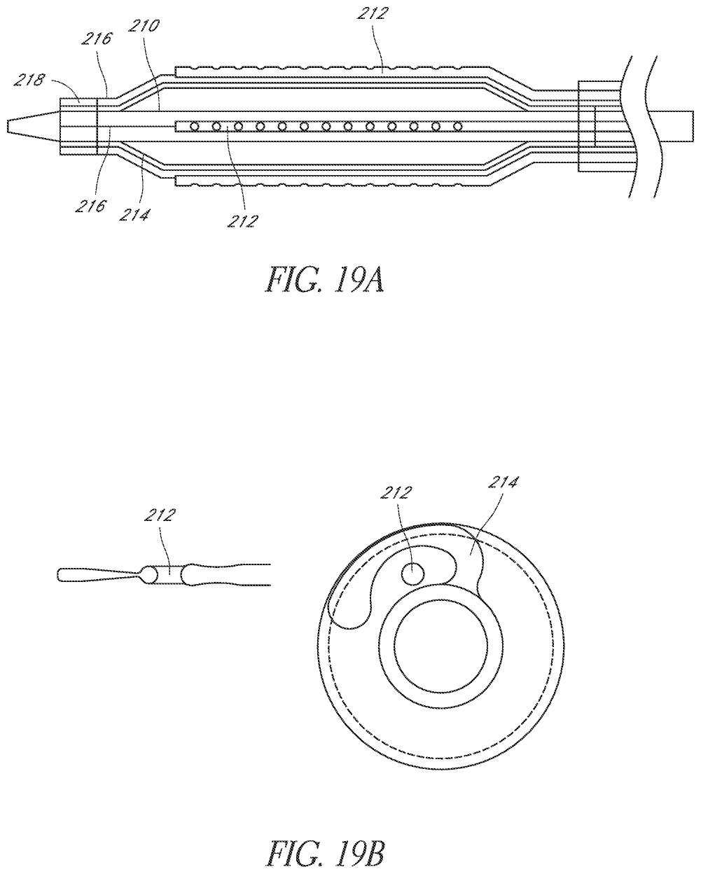

FIGS. 19A-19B schematically illustrates a delivery device 210 embodiment where the infusion tubes 212 are not bonded to the balloon 214, but are oriented and constrained radially by elongate structures, such as, for example, wires 216 running through their centers. The wires 216 can be gathered and integrated into the distal catheter tip 218. Instead of a central wire, the infusion tube 212 could also have a long tail cut on the distal end which could be organized and gathered at the distal catheter tip 218 similar to the wires. The infusion tubes 212 can be organized along the proximal shaft by sheathing them in an appropriate material such as PET heat shrink or running them through a multi-lumen extrusion. The delivery profile could be minimized by, for example, wrapping the infusion tubes underneath the pleats of the balloon, which could also serve as a mechanism to reduce the likelihood of thrombosis clogging the ports of the infusion tubes during delivery. The gathering of wires at the distal end can help integrate to native balloon cone and catheter tip. The multiport array of the infusion tubes 212 can have distinct delivery lumens. The porting can be progressive and/or flow balanced. The sheathing can gather and organize the lumens proximally. FIG. 19B shows the balloon wrap or pleat and the port profile minimized by overlap with pleated balloon 214. The cut profile of a single port or infusion tube 212 with distal extension to be gathered at the balloon cone 218 is also shown.

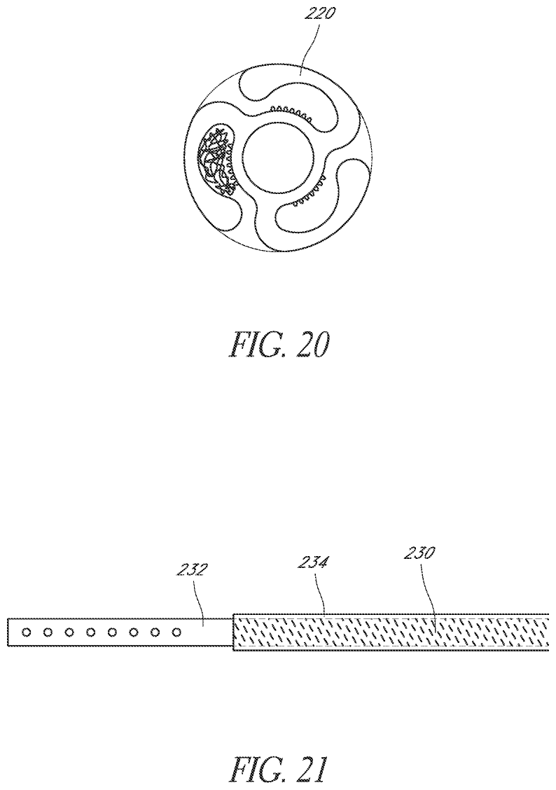

FIG. 20 schematically illustrates a delivery device 220 embodiment where the therapeutic agents, e.g., the MSCs, can be preloaded into the spaces underneath the balloon pleats. The balloon could have texture, wells, a reservoir, or other features to help hold the cells in place until balloon inflation. There could also be a thin film around the balloon to hold the cells in place until balloon inflation, at which point it could preferentially rupture to expose the MSC media. The delivery device 220 can have a film that is burst just prior to or during inflation. There can be texture or retention geometry on the balloon. The MSC can be pre-dosed within the dead volume inside pleats.

FIG. 21 schematically illustrates a delivery device 230 embodiment including a single inflation lumen made from a laser cut hypotube 232. The hypotube 232 could have a progressive interrupted spiral cut pattern to improve flexibility while also providing resistance to kinking and crushing. Cuts in the hypotube 232 could be sealed proximally with a flexible covering 234 such as PET heat shrink while porting for cell infusion could be left unrestricted. The laser cut hypotube 232 could also just be a short distal section that connects to a different tube or lumen for the majority of length to the hub. The hypotube 232 can have porting or holes. The hypotube 232 can have an articulated cut pattern that improves flexibility and trackability while providing hoop strength for support. The covering 234 can seal over the articulated section of hypotube 232.

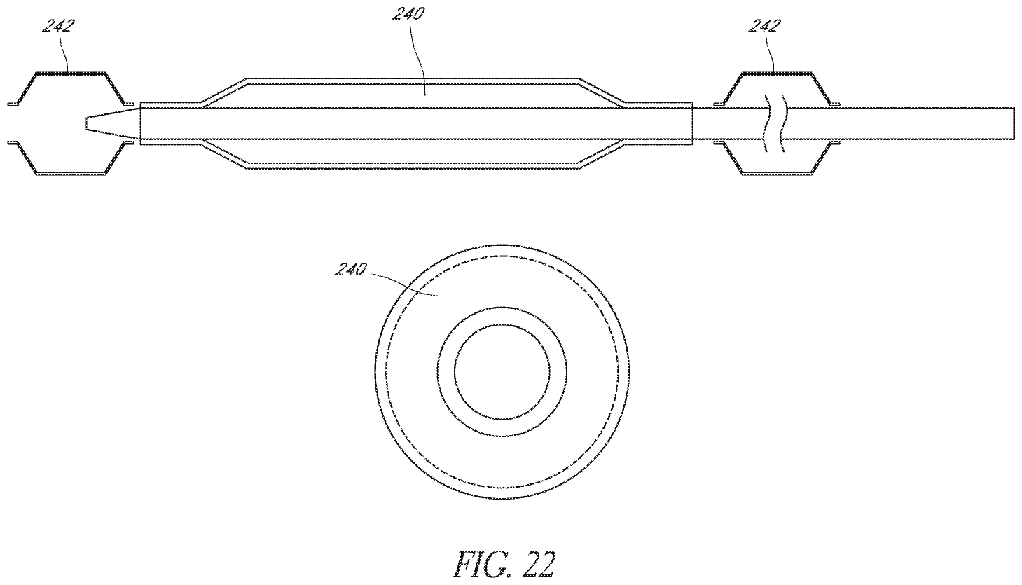

FIG. 22 schematically illustrates a delivery device 240 embodiment including dual expandable members, e.g., balloons 242 that can be positioned either or both distal and proximal to the target treatment area. This can help contain the therapeutic agents, e.g., MSCs for the duration of treatment. The dual balloon construct can be for sealing prior to stem-cell delivery in an isolated segment.

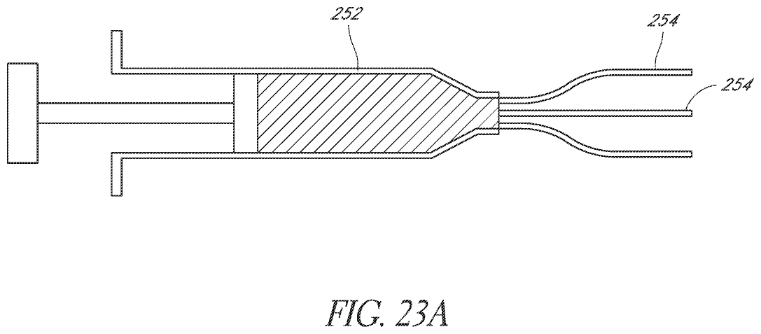

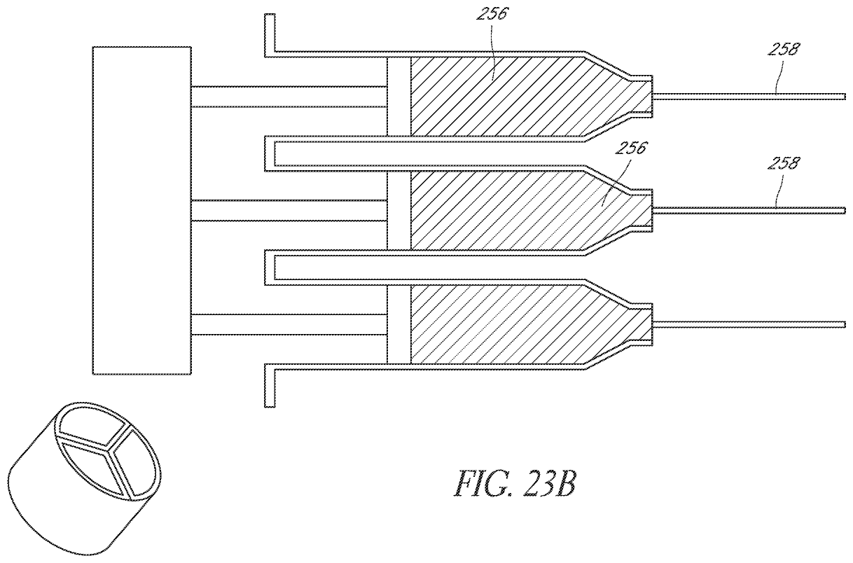

FIGS. 23A-23B schematically illustrates a delivery device embodiment and injection method. The therapeutic agent, e.g., MSC suspension could be held in a single syringe 252 that branches into multiple infusion lumens 254. In this case the cell solution can travel preferentially down the infusion lumen with the least resistance. The MSC suspension could also be held in separate syringes 256 that each connect to a single infusion lumen 258. This offers the advantage that the same volume of cell suspension will be injected through each infusion lumen. The handles of the three syringes could be connected or separate so that all three could be actuated in the same motion, or one at a time if the force is too high. The syringe barrels could be laid out in a linear or radial pattern. In some embodiments, three plungers that activated together can ensure equal volume in each infusion lumen. The three plungers can be linear or radial. An embodiment of three radial plungers for activation together is shown.

In some embodiments, the expandable member can be utilized to deliver therapeutic agents, including but not limited to cellular suspensions, in any desired anatomical location, including but not limited to vascular and non-vascular body lumens (including respiratory tract lumens such as the trachea and bronchi, GI tract lumens such as the esophagus, stomach, small intestine, large intestine, rectum, biliary tree; urinary tract lumens such as the ureters, bladder, and urethra; gynecological tract lumens such as the fallopian tubes, uterus, and vagina; and the like). Vascular body lumens can include the cerebral vasculature, coronary vasculature, and the peripheral vasculature, for example.

In some embodiments, a method for treatment of intimal hyperplasia can involve that the plaque layer within the vessel to be treated be either fractured or penetrated, and that a suspension of stem or non-stem cells be delivered beneath the plaque to the intima, subintimal space, media, adventitia, and/or periadventitial space. In some embodiments, systems and methods can be utilized to deliver SVF directly into the arterial wall in order to elicit a decreased neointimal hyperplasia response.

The fracturing of the plaque may be by inflation of a balloon, as is common in angioplasty, or by other mechanical means, such as the compression of a stent-like device to increase its diameter after positioning in the vessel. The fracturing of the plaque may be by conventional means, such as inflation of a balloon, prior to and separate from the introduction of stem cells, or may be combined in a single device which both fractures the plaque and subsequently introduces the cellular suspension. Penetration of the plaque may be by extension of spikes or similar structures after positioning a delivery device within the vessel. In either case, after fracturing or penetration, a suspension of stem cells is delivered under sufficient pressure to move the cells into the plaque layer and/or one or more of the selected structural layers of the vessel under treatment, where they remain after removal of the delivery device.

Another alternative embodiment of the delivery device (not shown) is a helical or double-helical arrangement of thin tubes having small apertures at intervals along the length of the tubes. Similar in appearance to a conventional stent, the device is inserted into a vessel using a catheter and positioned as desired. It is then drawn together to increase its diameter (by movement of a conic member, not shown), thereby being used to fracture the plaque layer and become deeply embedded in the plaque. Alternatively, the device may be formed from a memory metal which expands when freed from a constraining sheath, or when heated to body temperature. The catheter is then used to introduce a cell-based suspension to the tubes and this suspension exits the tubes through the small apertures. After introduction of the stem cells, the device is elongated to reduce its diameter, disengage it from the vessel wall, and it is withdrawn along with the catheter.

Yet another embodiment is a multi-lobed balloon, having small apertures or hollow spikes at the apex of each lobe where it contacts the vessel wall.

Yet another embodiment is a multi-lobed balloon, having one or more apertures within the space between the lobes, which space defines a channel that may be filled with a cellular suspension, to provide increased area of contact between the plaque layer and the suspension. In this embodiment, annular flanges, as described above, are used to contain the cellular suspension, and to direct blood flow through a central hollow lumen in the balloon, or through alternate channels on the outer surface of the balloon, in which case the annular flanges have apertures or notches that communicate with such channels or the central lumen.

Each balloon described above may be of multi-lumen, e.g., double-lumen design to allow inflation using a fluid that is separate and distinct from the cellular suspension to be delivered to the vessel wall.

For interventions directed to aneurysm therapy, the cell suspension delivery systems as disclosed herein can be capable of fully penetrating the venous wall may be used for delivery of cell therapy into the surrounding periarterial tissues and fat rather than directly into the aneurysmal arterial wall. Most arteries are adjacent to a paired vein. The cell suspension delivery device is inserted via the adjacent vein and deployed in the vein. When deployed, hollow spikes on the device penetrate through the venous wall into the tissue surrounding the aneurysmal artery. The cell preparation is delivered through the spikes after which the device is retrieved and removed. Alternatively, the cell preparation may be delivered via a catheter which is directed to contact and provide support against the venous wall. Once in contact with the wall, one or more needles, or spikes can be deployed and the injection delivered into the periarterial tissues.

In some embodiments, materials can be selected depending on the desired clinical result. Some non-limiting examples of materials that can be utilized include stainless steel hypotube and wire; Nitinol hypotube and wire; Heat shrink—PET, PTFE, FEP, Pebax; PTFE Liners—Free extruded or deposited on copper core; Nylon or PET balloons; Thermoplastic extrusions—single or multi-lumen; Polyimide tubing; and/or Cyanoacrylate and UV Cure adhesive.

In some embodiments, without limitation, the following fabrication techniques can be utilized for any number of features disclosed herein: Laser tube cutting; Mechanical hole drilling; Nitinol shape setting; Extrusion; Reflow/thermoplastic heat setting; Thermal bonding/heat staking; Balloon Molding; Injection Molding; Adhesive bonding—Cyanoacrylate, UV cure, and epoxy.

In some embodiments, a modular system with cell infusion tubes separate from balloons could allow clinicians to use a preferred angioplasty balloon.

In some embodiments, a syringe pump can be utilized to provide a steady infusion rate. However, a syringe pump is not required or used in some embodiments.

In some embodiments, a system can include a balloon including a series of discrete infusion lumens arranged along the exterior of an angioplasty balloon catheter to both augment the localized pressure effect during inflation and ensure targeted delivery of MSC solution directly into the sub-intima through distributed porting along the lumen. In some embodiments, a balloon catheter can include any number of the following features: infusion lumen and porting to be mechanically stable under high pressure (e.g. up to about or about 8, 9, 10, 11, 12, 13, 14, 15, 16, 17, 18, 19, 20 atm, or more, or ranges including any two of the foregoing values); fluid path and porting geometry compatible with therapeutic agent injection so as to maintain a high rate of cell viability; overall construction and delivery profile to be introducible through a sheath of about 6F in size, or about, at least about, or no more than about 4F, 5F, 6F, 7F, 8F, 9F, 10F, or ranges including any two of the foregoing values; system configured to maintain competitive trackability/target lesion access; catheter design compatible with guidewire including but not limited to standard 0.018″ guidewires; balloon to maintain a Rated Burst Pressure (RBP) of greater than or equal to about 14, 15, 16, 17, 18, 19, or 20 atm; radiopaque (RO) marker placement and visibility, balloon inflate/deflate time, and catheter working length can be, in some cases, consistent with conventional balloon catheters.

In some embodiments, examples of drugs that may be suitable for use in the methods and devices depending, on the specific disease being treated, and with consideration of the physical properties of the drug, include, without limitation, anti-restenosis, pro- or anti-proliferative, anti-inflammatory, anti-neoplastic, antimitotic, anti-platelet, anticoagulant, antifibrin, antithrombin, cytostatic, antibiotic, anti-enzymatic, anti-metabolic, angiogenic, cytoprotective, angiotensin converting enzyme (ACE) inhibiting, angiotensin II receptor antagonizing and/or cardioprotective drugs.

Examples of antiproliferative drugs include, without limitation, actinomycins, taxol, docetaxel, paclitaxel, sirolimus (rapamycin), biolimus A9 (Biosensors International, Singapore), deforolimus, AP23572 (Ariad Pharmaceuticals), tacrolimus, temsirolimus, pimecrolimus, zotarolimus (ABT-578), 40-O-(2-hydroxy)ethyl-rapamycin (everolimus), 40-O-(3-hydroxypropyl)rapamycin (a structural derivative of rapamycin), 40-O-[2-(2-hydroxy)ethoxy]ethyl-rapamycin (a structural derivative of rapamycin), 40-O-tetrazole-rapamycin (a structural derivative of rapamycin), 40-O-tetrazolylrapamycin, 40-epi-(N-1-tetrazole)-rapamycin, and pirfenidone.

Examples of anti-inflammatory drugs include both steroidal and non-steroidal (NSAID) anti-inflammatories such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus and pimecrolimus.

Examples of antineoplastics and antimitotics include, without limitation, paclitaxel, docetaxel, methotrexate, azathioprine, vincristine, vinblastine, fluorouracil, doxorubicin hydrochloride and mitomycin.

Examples of anti-platelet, anticoagulant, antifibrin, and antithrombin drugs include, without limitation, heparin, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as ANGIOMAX® (bivalirudin, from Biogen), calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic and 4-amino-2,2,6,6-tetramethylpiperidine-1-oxyl (4-amino-TEMPO).

Examples of cytostatic or antiproliferative drugs include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Examples of ACE inhibitors include, without limitation, quinapril, perindopril, ramipril, captopril, benazepril, trandolapril, fosinopril, lisinopril, moexipril and enalapril.

Examples of angiotensin II receptor antagonists include, without limitation, irbesartan and losartan.

Other therapeutic drugs that may find beneficial use herein include, again without limitation, alpha-interferon, genetically engineered endothelial cells, dexamethasone, antisense molecules which bind to complementary DNA to inhibit transcription, and ribozymes, antibodies, receptor ligands such as the nuclear receptor ligands estradiol and the retinoids, thiazolidinediones (glitazones), enzymes, adhesion peptides, blood clotting factors, inhibitors or clot dissolving drugs such as streptokinase and tissue plasminogen activator, antigens for immunization, hormones and growth factors, oligonucleotides such as antisense oligonucleotides and ribozymes and retroviral vectors for use in gene therapy, antiviral drugs and diuretics.

In other embodiments, a combination of any two, three, or other number of the foregoing drugs or other therapeutic agents can be utilized depending on the desired clinical result.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “accessing a vascular lumen” includes “instructing the accessing of a vascular lumen.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A cell-suspension delivery device comprising:
an outer deformable member comprising an external surface comprising a plurality of protrusions configured to be inserted into one or more layers of a body lumen when situated within the lumen of the body lumen, and an internal surface defining an internal cavity, each protrusion having a bore in fluid communication with the internal cavity and sized and configured to permit passage of cells suspended in a liquid medium from the internal cavity through the bore and into a wall of the body lumen.

2. The cell-suspension delivery device of claim 1, further comprising a catheter connected to the device, the catheter having a lumen in fluid communication with the internal cavity.

3. The cell-suspension delivery device of claim 1, wherein the outer deformable member comprises an expandable member.

4. The cell-suspension delivery device of claim 3, wherein the expandable member comprises an inflatable balloon.

5. The cell-suspension delivery device of claim 4, wherein the inflatable balloon comprises an outer balloon and an inner balloon.

6. The cell-suspension delivery device of claim 1, comprising at least one delivery tube comprising exit ports configured to deliver the cell suspension.

7. The cell-suspension delivery device of claim 6, wherein the at least one delivery tube comprises a round, flattened, or half-moon cross-section.

8. The cell-suspension delivery device of claim 6, wherein the at least one delivery tube is wrapped around the external surface of the outer deformable member.

9. The cell-suspension delivery device of claim 6, wherein the at least one delivery tube is helically wrapped around the external surface of the outer deformable member.

10. The cell-suspension delivery device of claim 6, wherein the at least one delivery tube is not bonded to the external surface of the outer deformable member.

11. The cell-suspension delivery device of claim 6, comprising a plurality of exit ports, wherein the exit ports progressively increase in size from a proximal end to a distal end of the outer deformable member.

12. The cell-suspension delivery device of claim 1, further comprising a flow conduit configured to allow continuous flow within the body lumen and through the delivery device.

13. The cell-suspension delivery device of claim 1, further comprising proximal and distal features configured to prevent the cell suspension from migrating outside of a target region.

14. The cell-suspension delivery device of claim 1, wherein the device does not comprise any drugs.

15. The cell-suspension delivery device of claim 1, wherein the expandable member is a lobed balloon.

16. The cell-suspension delivery device of claim 1, further comprising a proximal and distal barrier.

17. The cell-suspension delivery device of claim 1, wherein the plurality of protrusions comprise a straight injection needle.

18. The cell-suspension delivery device of claim 1, wherein the plurality of protrusions comprise a curved injection needle.

19. A cell-suspension delivery device comprising:
an expandable member comprising at least one ported infusion tube configured to be situated within the lumen of the body lumen, and an internal surface defining an internal cavity, each ported infusion tube having a bore in fluid communication with the internal cavity and sized and configured to permit passage of cells suspended in a liquid medium from the internal cavity through the bore and into a wall of the body lumen.

20. The cell-suspension delivery device of claim 19, wherein the at least one ported infusion tube is helical.

21. The cell-suspension delivery device of claim 19, wherein the at least one ported infusion tube has a semi-circular cross section.

22. The cell-suspension delivery device of claim 19, wherein the at least one ported infusion tube comprises elevated rims around a bore.

23. The cell-suspension delivery device of claim 19, wherein the at least one ported infusion tube comprises small needles extending radially outward.

24. The cell-suspension delivery device of claim 19, wherein the at least one ported infusion tube is not bonded to the balloon.

25. The cell-suspension delivery device of claim 19, wherein the at least one ported infusion tube is delivered straight and then rotated.

26. The cell-suspension delivery device of claim 19, wherein the at least one ported infusion tube has progressive hole sizing.

27. The cell-suspension delivery device of claim 19, further comprising a guide wire extending through a lumen of the at least one ported infusion tube.

28. The cell-suspension delivery device of claim 19, wherein the at least one ported infusion tube is retracted back to deliver cells suspended in the liquid medium

29. The cell-suspension delivery device of claim 19, wherein the at least one ported infusion tube comprises a cage.

30. The cell-suspension delivery device of claim 19, wherein the at least one ported infusion tube comprises a ported array configured to be delivered over a guide wire, wherein the expandable member is subsequently inserted.

31. The cell-suspension delivery device of claim 19, wherein the at least one ported infusion tube is configured to be nested within the pleats of the expandable member.

32. The cell-suspension delivery device of claim 19, wherein the at least one ported infusion tube comprises an articulated cut portion.

33. A cell-suspension delivery device comprising:
an expandable member comprising at least cutting or scoring elements configured to facilitate passage of cells suspended in a liquid medium into a wall of the body lumen.

34. The cell-suspension delivery device of claim 33, wherein the at least cutting or scoring elements is configured to pierce a compliant layer.

35. The cell-suspension delivery device of claim 33, wherein the at least cutting or scoring elements is configured to pierce a pressurized layer.

36. The cell-suspension delivery device of claim 33, wherein the at least cutting or scoring elements is configured to pierce an unpressurized layer.

37. The cell-suspension delivery device of claim 33, wherein the at least cutting or scoring elements is configured to score the vessel wall.

38. The cell-suspension delivery device of claim 33, wherein the at least cutting or scoring elements is configured to guide the cells suspended in to liquid medium into tissue.

39. The cell-suspension delivery device of claim 33, further comprising a dual balloon construct to isolate the delivery between a distal and proximal balloon.

40. (canceled)
